(19) United States
(12) Patent Application Publication
(10) Pub. No.: US 2011/0003780 A1
(43) Pub. Date: Jan. 6, 2011

(54) HYDROGEN CHLORIDE SALT OF A SUBSTITUTED 5-OXAZOL-2-YL-QUINOLINE COMPOUND AND A PROCESS FOR THE PRODUCTION THEREOF

(75) Inventor: Man Zhu, Clark, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: SCHERING CORPORATION, Kenilworth, NJ (US)

(21) Appl. No.: 12/668,191

(22) PCT Filed: Jul. 3, 2008
(86) PCT No.: PCT/US08/08260

§ 371 Date: Jan. 7, 2010

Related U.S. Application Data
(60) Provisional application No. 60/959,479, filed on Jul. 10, 2007.

(51) Int. Cl. A61K 31/56; C07D 413/14; A61K 31/4709; A61P 11/00; A61P 11/06

(52) U.S. Cl. 514/171; 546/167; 514/314

(57) ABSTRACT

The present invention relates to the compound of the Formula I:

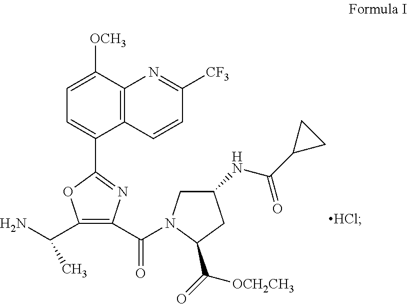

and to methods of treating upper and lower obstructive airway diseases using said compound, to formulations comprising it, and to a particular crystalline form and processes of synthesis of the crystalline form.

IM=105109

### CROSS REFERENCE TO RELATED APPLICATIONS

This application is based on and claims the priority of U.S. Provisional Patent Application No. 60/959,479, filed Jul. 10, 2007, the description of which is incorporated herein by reference in its entirety.

### FIELD OF THE INVENTION

The present invention relates to the hydrogen chloride salt of 1-[[5-(1(S)-aminoethyl)-2-[8-methoxy-2-(trifluoromethyl)-5-quinolyl]-4-oxazolyl]carbonyl]-4(R)-[(cyclopropyl-carbonyl)amino]-L-proline, ethyl ester, pharmaceutical compositions comprising said salt, and methods of treating upper and lower obstructive diseases of the airways by inhalation of said salt.

### BACKGROUND OF THE INVENTION

Identification of any publication, patent, or patent application in this section or any section of this application is not an admission that such publication is prior art to the present invention.

Phosphodiesterases are known to regulate cyclic AMP, and phosphodiesterase 4 (PDE4) has been shown to be the predominant regulator of cyclic AMP in respiratory smooth muscle and inflammatory cells. Inhibitors of PDE4 are useful in treating a variety of diseases, including allergic and inflammatory diseases, diabetes, central nervous system diseases, pain, and viruses that produce TNF.

Amino-substituted quinolyl PDE4 inhibitors are disclosed in U.S. Pat. No. 5,804.588; sulfonamide-substituted quinolyl PDE4 inhibitors are disclosed in U.S. Pat. No. 5,834,485; and (benzo-fused)heteroaryl-substituted PDE4 inhibitors are disclosed in U.S. Pat. No. 6,069,151, Oxazolyl-substituted quinolyl PDE4 inhibitors are disclosed in PCT/US2005/017134. A process for the preparation of the compound of Formula Ia, 1-[]5-(1(S)-aminoethyl)-2-[8-methoxy-2-(trifluoromethyl)-5-quinolyl]-4-oxazolyl]carbonyl]-4(R)-[(cyclopropyl-carbonyl)amino]-L-proline, ethyl ester, is described in published U.S. Patent Application 2006/0106062, published May 18, 2006, which is incorporated herein in its entirety by reference.

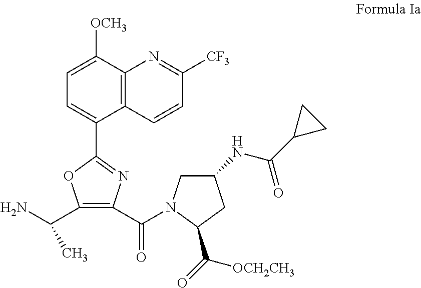

### SUMMARY OF THE INVENTION

One aspect of the present invention is the hydrogen chloride salt of 1-[]5-(1(S)-aminoethyl)-2-[8-methoxy-2-(trifluoromethyl)-5-quinolyl]-4-oxazolyl]carbonyl]-4(R)-[(cyclopropyl-carbonyl)amino]-L-proline, ethyl ester, the compound of Formula I:

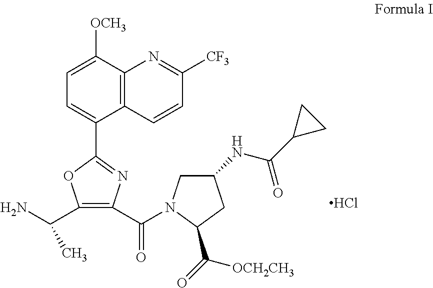

exhibiting a powder x-ray diffraction pattern (PXRD pattern) substantially similar to that of FIG. 1, which has as its four most characteristic peaks appearing at diffraction angles of 7.9, 15.6, 18.5, and 23.4 degrees 2θ.

Another aspect of the present invention is crystalline form of the compound of Formula I (hydrogen chloride salt of the compound of Formula Ia) having a PXRD pattern which has it's eight most characteristic peaks appearing at 7.9, 13.8, 15.6, 17.2, 18.5, 19.7, 23.4, and 27.2 degrees 2θ.

Another aspect of the present invention is a crystalline form of the compound of Formula I (hydrogen chloride salt of the compound of Formula Ia) having a PXRD pattern which has it's twelve most characteristic peaks appearing at 7.9, 8.5, 13.8, 15.6, 17.2, 18.5, 19.7, 23.4, 27.2, 27.6, 29.3 and 30.9 degrees 2θ.

Another aspect of the present invention is a method of treating upper or lower obstructive diseases of the airways in a patient in need of such treatment comprising administering to said patient by inhalation an effective amount of a medicament comprising the compound of Formula I having a PXRD containing peaks at diffraction angles of 7.9, 15.6, 18.5, and 23.4 degrees 2θ, optionally in combination with at least one additional agent useful for treating upper or lower obstructive diseases of the airway, preferably an additional agent selected from beta-agonists, muscarinic antagonists or corticosteroids.

Another aspect of the present invention is the provision of an inhalable pharmaceutical composition comprising an effective amount of the compound of Formula I having a PXRD containing peaks at diffraction angles of 7.9, 15.6, 18.5, and 23.4 degrees 2θ, optionally in combination with at least one additional agent useful for treating upper or lower obstructive diseases of the airway, preferably an additional agent selected from beta-agonists, muscarinic antagonists or corticosteroids.

Another aspect of the present invention is a process for preparing the crystalline form of the compound of Formula I having a PXRD containing peaks at diffraction angles of 7.9, 15.6, 18.5, and 23.4 degrees 2θ, the process comprising:
a) providing an ethanol solution of the compound of Formula Ia:

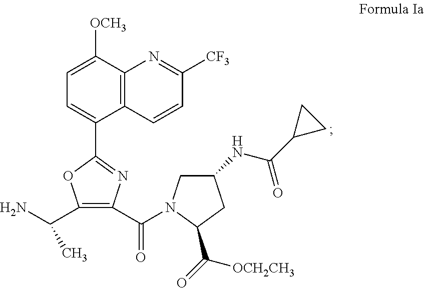

b) reacting the solution from Step “a” with an isopropanol solution of HCl at refluxing temperature;
c) cooling the mixture provided in Step “b” to form a salt precipitate; and
d) optionally isolating the precipitate by filtering the mixture then washing and drying the resulting solid to yield the compound of Formula I (a hydrogen chloride salt of the compound of Formula Ia),

Other aspects and advantages of the present invention will become apparent from following Detailed Description.

### BRIEF DESCRIPTION OF THE DRAWINGS

FIG. 1 is a graph of a powder x-ray diffraction (PXRD) pattern of the crystalline form of the compound of Formula I, generated using an X-ray diffractometer. The graph plots the intensity of the peaks as defined by counts per second versus the diffraction angle 2 θ in degrees.

FIG. 2 is a plot of the thermal analysis of the crystalline form of the compound of Formula I generated by Differential Scanning Calorimetry (DSC).

### DETAILED DESCRIPTION

The free base compound of Formula Ia, having the structure:

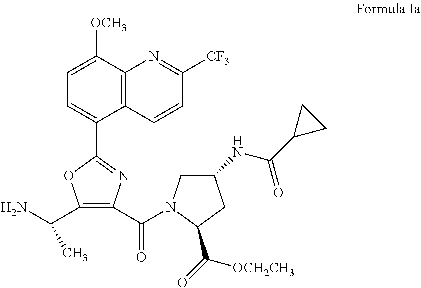

is described in US 2006/0106062 A1, published May 18, 2006 (the '062 publication), which application is incorporated herein by reference in its entirety. In particular, the '062 publication describes a batch process for the preparation of the compound of Formula Ia on pages 83 to 86 (preparative Examples 5 to 7, in preparation of the example compound 26-347, which is illustrated on page 193), which process is incorporated by reference herein in its entirety. A process for preparing the compound of Formula Ia is also described in an application filed herewith on Jul. 10, 2007 under attorney's docket no. CD06670L01US, which is incorporated herein by reference.

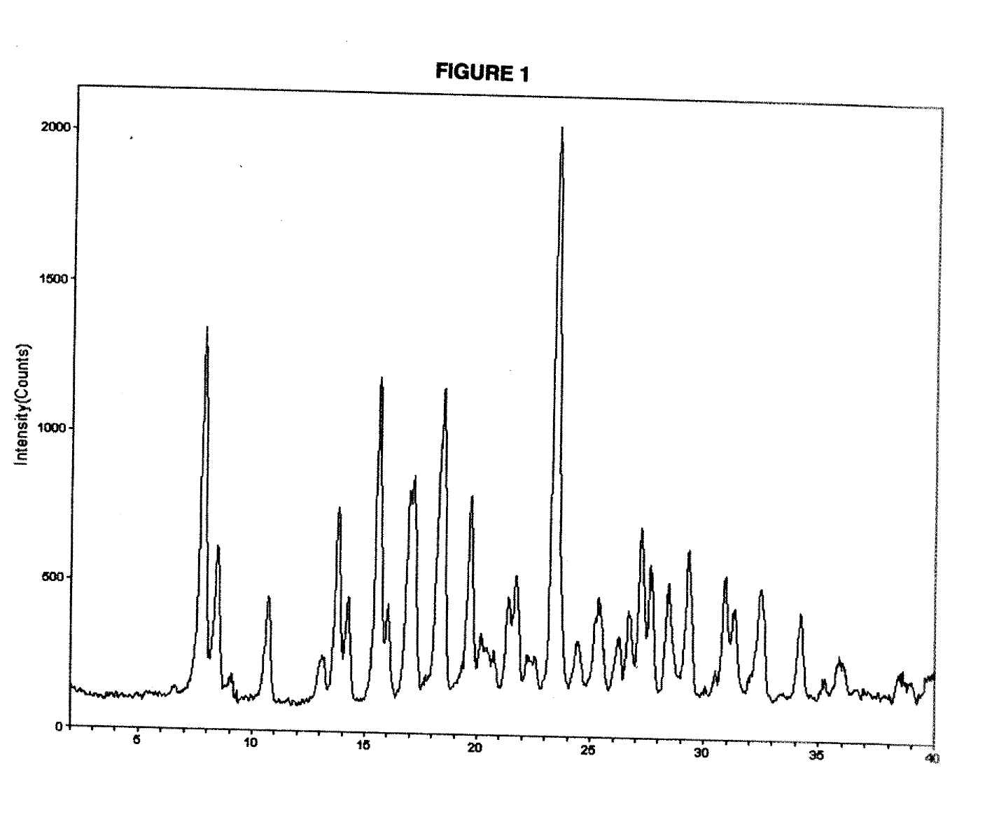

The compound of Formula I is a crystalline hydrogen chloride salt form of the compound of Formula Ia. The compound of Formula I can be provided in a crystalline form which exhibits a PXRD substantially similar the X-ray powder pattern shown in FIG. 1, and has its four most characteristic peaks at diffraction angles of 7.9, 15.6, 18.5, and 23.4 degrees 2θ.

It is believed that this crystalline form of the compound of Formula I demonstrates superior stability over some other salts, and improved solubility without unacceptable hygroscopic properties. Accordingly, it is believed that the compound of Formula I may provide medicaments having increased shelf life and improved solubility, and thereby improved bioavailability, when compared to some other salts of the compound of Formula Ia. Since the intended use of the compound of Formula I is as a therapeutically active pharmaceutical agent, salt forms of the compound of Formula Ia having notable stability and bioavailability are of great interest.

The unique crystalline material comprising the compound of Formula I can be prepared as described herein in the Examples, in particular step 5 of Example 2.

As used throughout the specification, the following terms, unless otherwise indicated, shall be understood to have the following meanings:

“Patient” includes both human and other animals.

“Mammal” includes humans and other mammalian animals.

“Alcohol” means an organic compound containing a hydroxyl group (—OH).

“Excipient” means an essentially inert substance used as a diluent or to give form or consistency to a formulation.

“Effective” or “therapeutically effective” is meant to describe a polymorph of a compound or a composition of the present invention effective as a PDE4 inhibitor and thus producing the desired therapeutic, ameliorative, inhibitory or preventative effect. “Effective amount” or “therapeutically effective amount” is meant to describe an amount of polymorph or a composition of the present invention effective as a PDE4 inhibitor and thus producing the desired therapeutic, ameliorative, inhibitory or preventative effect.

Upper and lower airway obstructive disease treated by the compound of Formula I include asthma, COPD (chronic obstructive pulmonary disease), chronic bronchitis, cystic fibrosis, allergic rhintis, non-allergic rhinitis, rhinosinusitis, adult respiratory disease, acute respiratory distress syndrome, respiratory viruses, cough, interstitial pneumonitis, chronic sinusitis, airflow obstruction, airway hyperresponsiveness (i.e., airway hyperreactivity), bronchiectasis, bronchiolitis, bronchiolitis obliterans (Le., bronchiolitis obliterans syndrome), dyspnea, emphysema, hypercapnea, hyperinflation, hypoxemia, hyperoxia-induced inflammations, pulmonary fibrosis, pulmonary hypertension, small airway disease, wheeze and colds.

The compound of Formula I is preferably useful in treating asthma, CORD, cough, airflow obstruction, airway hyperresponsiveness (Le., airway hyperreactivity), bronchiolitis, chronic bronchitis, emphysema, pulmonary fibrosis, pulmonary hypertension, small airway disease, wheeze and allergic rhinitis.

More preferably, the compound of Formula I is useful for treating COPD and asthma.

Other agents for treating an obstructive airway disease (e.g., CORD or asthma) for use in combination with the compound of Formula I are selected from the group consisting of: steroids (e.g. glucocorticoids), 5-lipoxygenase inhibitors, β-2 adrenoceptor agonists, α-adrenergic receptor agonists, muscarinic M1 antagonists, muscarinic M3 antagonists, muscarinic M2 antagonists, LTB4 antagonists, cysteinyl leukotriene antagonists, bronchodilators, PDE4 inhibitors, elastase inhibitors, MMP inhibitors, phospholipase A2 inhibitors, phospholipase D inhibitors, histamine H1 antagonists, histamine H3 antagonists, dopamine agonists, adenosine A2 agonists, NK1, NK2 and NK3 antagonists, GABA-b agonists, nociceptin agonists, expectorants, mucolytic agents, decongestants, mast cell stabilizers, antioxidants, anti-IL-8 antibodies, anti-IL-5 antibodies, anti-IgE antibodies, anti-TNF antibodies, IL-10, adhesion molecule inhibitors, growth hormones and other PDE4 inhibitors,

Non-limitative examples of antihistamines that can be used in combination with the compound of Formula I include astemizole, azatadine, azelastine, acrivastine, brompheniramine, certirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, equitazine, mianserin, noberastine, meclizine, norasternizole pirumast pyrilamino, promethazine, terfenadine, tripeiennamnee temelastine, trirneprazine and triprolidine.

Non-limitative examples of histamine H₃ receptor antagonists include: thioperamicle, impromidine, burimamide, clobenpropit, irnpentamine, mifetidine, S-sopromidine, R-sopromidine, SKF-91486, GR-175737, GT-2016, UCL-1199 and clozapine. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West et al., “Identification of Two-H₃-Histamine Receptor Subtypes,” Molecular Pharmacology, Vol. 38, pages 610-613 (1990).

The term “leukotriene inhibitor” includes any agent or compound that inhibits, restrains, retards or otherwise interacts with the action or activity of leukotrienes. Non-limitative examples of leukotriene inhibitors include montelukast and its sodium salt; 1-(((R)-(3-(2-(6,7-difluoro-2-quinolinyl)ethenyl)phenyl)-3-(2-(2-hydroxy-2-propyl) phenyl)thio)methylcyclopropaneacetic acid, and its sodium salt, described in U.S. Pat. 5,270,324; 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclo-propaneacetic acid, and its sodium salt, described in U.S. Pat. No. 5,472,964; pranlukast; zafirlukast; and [2-[[2(4-tert-butyl-2-thiazolyl)-5-benzofuranyl]oxymethyl]phenyl] acetic acid, described in U.S. Pat. No. 5,296,495.

Non-limitative examples of β-adrenergic receptor agonists include: albuterol, bitolterol, isoetharine, mataproterenol, perbuterol, salmeterol, terbutaline, isoproterenol, ephedrine and epinephrine. Non-limitative examples of α-adrenergic receptor agonists include arylalkylamines, (e.g., phenylpropanolamine and pseudephedrine), imidazoles (e.g., naphazoline, oxymetazoline, tetrahydrozoline, and xylometazoline), and cycloalkylamines (e.g., propylhexedrine).

A non-limitative example of a mast cell stabilizer is nedocromil sodium. A non-limitative example of an expectorant is guaifenesin. Non-limitative examples of decongestants are pseudoephedrine, phenylpropanolamine and phenylephrine.

Non-limitative examples of other PDE4 inhibitors include roflumilast, theophylline, rolipram, piclamist, cilomilast and CDP-840. Examples of steroids include prednisolone, fluticasone, triamcinolone, beclomethasone, mometasone, budisamide, betamethasone, dexamethasone, prednisone, flunisolide and cortisone.

Non-limitative examples of NK₁, NK₂ and NK₃ tachykinin receptor antagonists include CP-99,994 and SR 48968. Non-limitative examples of muscarinic antagonists include ipratropium bromide and tiatropium bromide.

Non-limiting examples of GABA_{B} agonists include baclofen and 3-aminopropyl-phosphinic acid. Dopamine agonists include quinpirole, ropinirole, pramipexole, pergolide and bromocriptine,

“5-lipoxygenase inhibitors” include any agent or compound that inhibits, restrains, retards or otherwise interacts with the enzymatic action of 5-lipoxygenase. Non-limitative examples of 5-lipoxygenase inhibitors include zileuton, docebenone, piripost, ICI-D2318, and ABT 761.

The compound of Formula I was prepared by the procedure outlined in Schemes 1 or 2 and detailed in the following Examples 1 or 2, In Example 1 and elsewhere in the application, Et means ethyl, Me means methyl, THF is tetrahydrofuran, DMF is N,N-dimethylformamide, t-BOC and BOC mean t-butoxycarbonyl, RT is room temperature, HATU is N-[(dimethylamino)-1H-1,2,3-triazolo[4,5-b]pyridin-1-ylmethylene]-N-methylmethanaminium hexafluorophosphate N-oxide, MS means mass spectra or mass spectrum. In the application, ETOH means ethanol, NMR means Nuclear Magnetic Resonance, DMSO means dimethyl sulfoxide, Et₃N means triethylamine, NaHMDS is sodiumbis(trimethylsilyl)amide, HOBT is hydroxybenztriazole, IPA is isopropanol (isopropyl alcohol), EDO HCl is 1-ethyl-3-[3-dimethylamino)propyl]-carbodimide hydrochloride, NMP is N-methylpyrrolidinone, ca is circa (about), KF is Karl Fisher, and EtOAc is ethyl acetate.

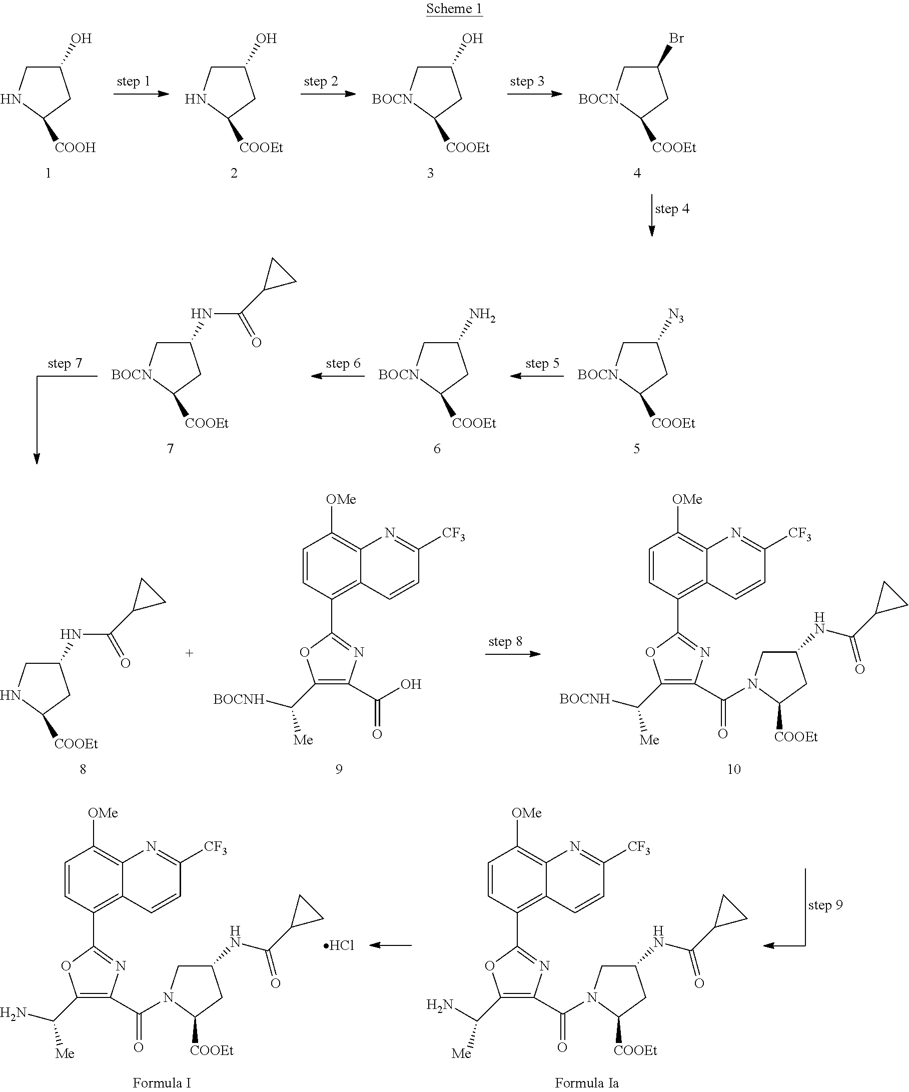

### Example 1

### Step 1:

To a mechanically stirred suspension of compound 1 (100.6 g, 0.767 mol) in EtOH (1000 ml) and cooled to 0° C. was added SOCl₂ (136.9 g, 1.15 mol, 84.0 ml) dropwise via addition funnel such that the internal temperature was <15° C. The reaction mixture was heated at reflux for 2.5 h, then cooled to 0° C. Ether (1000 ml) was added, and a white solid precipitated. The solid was isolated by vacuum filtration and washed with ether. The product 2 (HCl salt) was dried in a vacuum oven to give 146.3 g (97%) of a white solid. MS (M+1): m/e 160. ¹H-NMR (DMSO) δ 1.25 (t, 3H), 2.05 (m, 1H), 2.20 (m, 1H), 3.05 (d, 1H), 3.40 (dd, 1H), 4.20 (q, 2H), 4.45 (m, 2H), 5.65 (broad s, 1H).

### Step 2:

To a solution of compound 2 (HCl salt, 146.2 g, 0.747 mol) dissolved in CH₂Cl₂ (1600 ml) and EtOH (100 ml) and cooled to 0° C. was added Et₃N (113.4 g, 1.12 mol, 156.2 ml). t-BOC anhydride (195.6, 0.90 mol) was added portionwise. The reaction mixture was stirred at 0° C. for 15 min, then at RT for 16 h. The resulting mixture was concentrated to ˜800 ml volume and washed with water. The organic solution was dried (MgSO₄), filtered, and concentrated. Purification by silica gel chromatography (eluant: 20% EtOAc—CH₂Cl₂) gave the product 3 (193.7 g, 100%) as a yellow oil. MS (M+Na): m/e 282. ¹H-NMR (CDCl₃) δ 1.30 (t, 3H), 1.45 (s, 9H), 1.75 (m, 1H), 2.10 (m, 1H), 2.30 (m. 1H), 3.45 and 3.55 (d, 1H for two rotamers), 3.65 (dd, 1H), 4.25 (m, 2H), 4.40 and 4.45 (t, 1H for two rotamers), 4.55 (broad s, 1H).

### Step 3:

To a solution of compound 3 (36.5 g, 0.141 mol) and triphenyl phosphine (46.2 g, 0.176 mol) dissolved in dry THF (1000 ml) and cooled to 0° C. was added diethyl azodicarboxylate (30.7 g, 0,176 mol) dropwise via addition funnel. The reaction mixture was stirred at 0° C. for 5 min, then LiBr (61.1 g, 0.704 mol) was added in one portion. The resulting mixture was stirred at RT for 16 h. The solvent was evaporated, water (1500 ml) was added, and the aqueous solution was extracted with CH₂Cl₂. The combined organic extracts was dried (MgSO₄), filtered, and concentrated. Purification by silica gel chromatography (eluant: 2% EtOAc—CH₂Cl₂ to 5% EtOAc CH₂Cl₂) gave the product 4 (31.8 g, 70%) as a yellow oil. MS (M+1): m/e 322 and 324. ¹H-NMR (CDCl₃) δ 1.30 (m, 3H), 1.45 and 1.50 (s, 9H for two rotamers), 2.45 (m, 1H), 2.85 (m, 1H), 3.75 (m, 1H), 4.05-4.40 (m, 5H).

### Step 4:

To a solution of compound 4 (41.2 g, 0.128 dissolved in dry DMSO (300 ml) as added NaN₃ (9.15 g, 0.141 mol). The reaction mixture was stirred at RT for 16 h. Water (300 ml) was added, and the aqueous solution was extracted with ether. The combined, organic extracts was dried (MgSO₄), filtered, and concentrated to give the product 5 (36.4 g, 100%) as an oil. MS (M+Na): m/e 307. ¹H-NMR (CDCl₃) δ 1.30 (t, 3H), 1.45 and 1.50 (s, 9H for two rotamers), 2.20 (m, 1 H), 2.35 (m, 1H), 3.50 and 3.60 (m, 1H for two rotamers), 3.75 (m, 1H), 4.15-4.45 (m, 4H).

### Step 5:

To a solution of compound 5 (36.4 g, 0.128 mol) dissolved in THF (800 ml) was added 10% palladium on carbon catalyst (10.0 g). The reaction mixture was shaken on a Parr shaker under 40 psi of hydrogen pressure for 16 h. The catalyst was removed by filtration and washed with isopropanol. The filtrate was concentrated. Purification by silica gel chromatography (eluant: CH₂Cl₂ then 10% MeOH with NH₃—CH₂Cl₂) gave the product 6 (24.2 g, 73%) as a light gray solid. MS (M+1): m/e 259. ¹H-NMR (CDCl₃) δ 1.30 (t, 3H), 1.45 and 1.50 (3, 9H for two rotamers), 2.00 (m, 1H), 2.15 (m, 1H), 3.10 and 3.20 (m, 1H for two rotamers), 3.70 (m, 2H), 4.20 (m, 2H), 4.35 and 4.40 (m, 1H for two rotamers).

### Step 6:

To a solution of compound 6 (12.0 g, 0.0464 mol) dissolved in dry CH₂Cl₂ (300 ml) was added Et₃N (9.4 g, 0.093 mol, 13.0 ml) then cyclopropanecarbonyl chloride (5.3 g, 0.051 mol, 4.64 ml). The reaction mixture was stirred at RT for 16 h. Water (200 ml) was added, and the aqueous solution was extracted with CH₂Cl₂. The combined organic extracts was dried (MgSO₄), filtered, and concentrated. Purification by silica gel chromatography (eluant: 5% MeOH with NH₃—CH₂Cl₂) gave the product 7 (14.3 g, 94%) as an oil. MS (M+Na): m/e 349. ¹H-NMR (CDCl₃) δ 0.75 (d, 2H), 1.00 (broad s, 2H), 1.30 (t, 3H), 1.35 (m, 1H), 1.45 and 1.50 (s, 9H for two rotamers), 2.25 and 2.30 (m. 2H for rotamers), 3.30 and 3.45 (dm, 1H for rotamers), 3.80 (m, 1H). 4.15-4.45 (m, 3H), 4.55 (m, 1H), 5.95 and 6.10 (broad singlet, 1H for rotamers).

### Step 7:

To a solution of compound 7 (40.0 g, 0.123 mol) dissolved in CH₂Cl₂ (550 ml) was added 4 N HCl in dioxane (153 ml, 0.613 mol). The reaction mixture was stirred at RT for 4 h then concentrated to give the product 8 (32.2 g, 100%) as a colorless foam. MS (M+1): m/e 227. ¹H-NMR (CDCl₃) δ 0.75 (d, 2H), 0.90 (m, 2H), 1.30 (t, 3H), 1.55 (m, 1H), 2.35 (m, 1H), 2.55 (m, 1H), 3.70 (m, 2H), 4.25 (m, 2H), 4.75 (m, 2H), 8.35 (d, 1H), 9.05 (broad s, 1H).

### Step 8:

To a mixture of compound 8 (5.5 g, 20.8 mmol) and carboxylic acid 9 (10.0 g, 20.8 mmol) in dry DMF (300 ml) was added 3 A sieves (10.0 g), Et₃N (6.3 g, 62.3 mmol, 8.7 ml), then HATU (15.8 g, 41,6 mmol). The reaction mixture was stirred at RT for 21 h then the solvent was concentrated, Water (400 ml) was added, and the aqueous solution was extracted with CH₂Cl₂. The combined organic extracts were dried (MgSO₄), filtered, and concentrated. Purification by silica gel chromatography (eluant: 20% EtOAc—CH₂Cl₂ to 60% EtOAc—CH₂Cl₂) gave the product 10 (14.0 g, 98%) as a colorless foam. MS (M+1): m/e 690. See FIG. 3 for the NMR spectrum.

### Step 9:

To a solution of compound 10 (42.1 g, 0.061 mol) dissolved in CH₂Cl₂ (600 ml) and cooled to 0° C. was added 4 N HCl in dioxane (76 ml, 0.305 mol). The reaction mixture was then stirred at RT for 5 h and then concentrated. The crude product was dissolved in 1:1 EtOH:H₂O (120 ml) and made basic (pH=9-10) with 25% aqueous NaOH. CH₂Cl₂ (700 ml) was added, and the reaction mixture was stirred until all solids dissolved. The layers were separated, and the aqueous solution was extracted with CH₂Cl₂. The combined organic extracts was washed with brine, dried (MgSO₄), filtered, and concentrated. Additional CH₂Cl₂ was added, and the mixture was concentrated again. Ether was added, and the mixture was concentrated to give compound of Formula Ia (34.4 g, 96%) as a light yellow solid. MS (M+1): m/e 590.

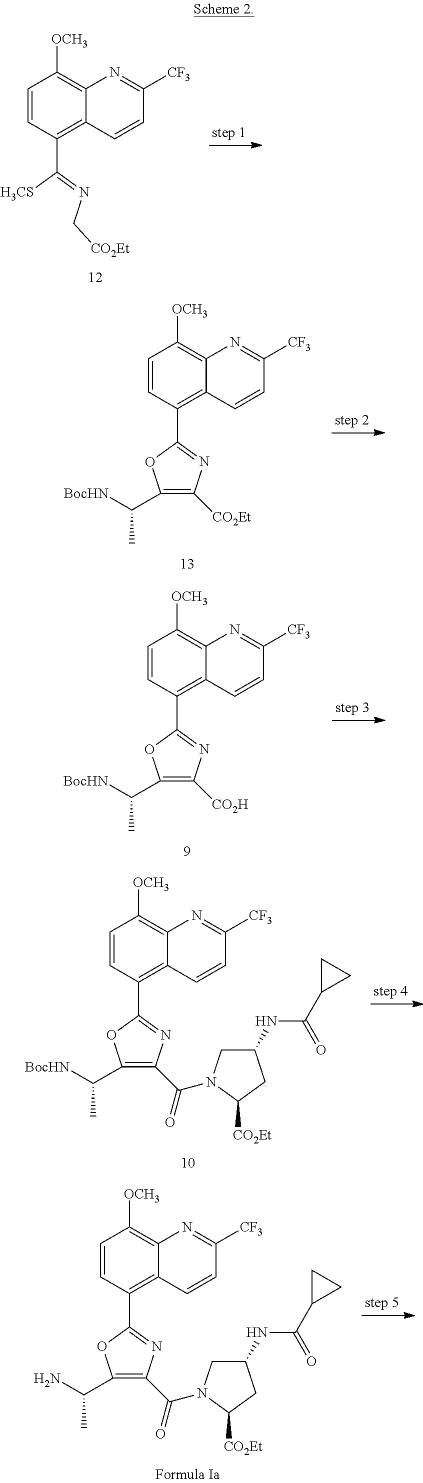

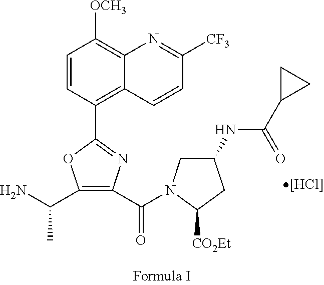

### Example 2

### Step 1:

Into a 50 L Hastelloy reactor equipped with a thermocouple, N₂ inlet and feed tank was charged 8.8 kg (46.5 moles, 2 eq) of (S)-2-tert-butoxycarbonylamino-propionic acid and 90 liters dry tetrahydrofuran (THF, KF <0.05%) to dissolve the acid. Into the reactor was slowly charged 8.5 kg (46.9 moles, 2 eq) of dicyclohexylamine over about 30 minutes while maintaining the temperature or the reaction mixture from about [−5° C.] to about [+5° C.]. The mixture was agitated for about 15 minutes while maintaining the reaction mixture from about [−5° C.] to about [+5° C.]. At the end of the agitation period, 5.7 kg (47.3 moles, 2 eq) of trimethylacetylchioride was charged into the reaction mixture over about 30 minutes while maintaining the temperature of the reaction mixture from about [−5° C.] to about [+5° C.]. The mixture was agitated for about 3 hours while maintaining the temperature of the reaction mixture from about [−5° C.] to about [+5° C.]. At the end of the agitation period the reactor was charged with 27 liters of heptane, followed by 4.5 kg of celite. The reaction mixture was filtered under N₂, and the filter cake thus obtained was washed with 30% v/v THF in heptane. The fiitrate and washes were combined and concentrated by distillation under vacuum to a batch volume of about 36 liters. The concentrated reaction mixture was diluted with 27 liters of THF and the temperature of the mixture was adjusted to a temperature between 20° C. and 30° C. A sample of the reaction mixture was tested for residual water by Karl Fischer titration and found to be less than about 0.06 ppm. The mixed anhydride/THF solution thus obtained was used in the next step without further purification.

Into a 50 gallon glass lined reactor equipped with a thermocouple, N₂ inlet and feed tank was charged 9.0 kg (23.3 moles, 1 eq) of the compound (12) and 126 liters dry tetrahydrofuran (THF, KF <0.05%), with agitation to dissolve the compound of Formula (12). The reaction mixture was concentrated by distillation at 1 atmosphere to a batch volume of about 81 liters. The temperature of the concentrated reaction mixture was adjusted and maintained at a temperature of from [−60° C.] to [−70° C.]. Into the reaction mixture was charged NaHMDS (2M in THF, 2.70 kg, 5.9 moles, 0.25 eq) over about 15 minutes while maintaining the temperature of the reaction mixture. At the end of the addition period the reaction mixture was agitated for 5 minutes while continuing to maintain the temperature. Following agitation the reaction mixture was charged over a period of 15 minutes with the mixed anhydride/THF solution prepared previously (0.83 kg active anhydride, 3.2 moles, 0.14 eq) while maintaining the reaction mixture at a temperature of from [−60° C.] to [−70° C.], following which the mixture was agitated for about 10 minutes while continuing to maintain the temperature. Two additional charges of (NaHMDS 2M in THF), each followed by a charge of the mixed anhydride/THF solution were preformed, followed by five (5) additional charges of the anhydride/THF solution for a total of eight (8) sets of charges or until the conversion is ≧70%. With each charge an agitation period was carried out and the temperature of the reaction mixture was maintained at a temperature of from [−60° C.] to [−70° C.] throughout the charging and agitating period. Charging NaHMDS (2M in THF) followed by the mixed anhydride/THF solution in the same ratio based on the amount of starting material remaining was until the a conversion of ≧94% was observed. When the conversion exceeded about 94%, the reaction mixture was transferred slowly, over about 15 minutes, to an aqueous solution of 13.5 kg KH₂PO₄ dissolved in 90 liters H₂O whilst maintaining the batch temperature below 30° C. At the end of the addition period, to the resulting mixture was charged 59 liters ethyl acetate and the mixture was agitated for about 15 minutes, then the layers were allowed to settle. The layers were separated and the aqueous layer extracted with 45 liters ethyl acetate. The ethylacetate wash was separated and the combined wotj the separated organic layer. The combined organics were washed two times with 32 liters 10% aqueous w/v NaCl then concentrated at 1 atmosphere to a volume of about 45 liters. To the concentrate was charged 90 liters methyltertbutylether (MTBE) and the mixture was concentrated at 1 atmosphere to a batch volume of about 54 liters. To the concentrate was charged 45 liters methyltertbutylether followed by 108 liters of heptane while maintaining the reaction mixture at a temperature of from 55° C. to 65° C. The temperature of the reaction mixture was adjusted to a temperature of from 45° C. to 55° C. and agitate for about 30 minutes while maintaining the temperature. At the end of the agitation period the temperature of the reaction mixture was adjusted to a temperature of from [−5° C.] to [+5° C.] over a 1 hour period and agitated for an additional 30 minutes while maintaining the temperature. At the end of the agitation period the reaction mixture was filtered and the filter cake thus obtained was washed with 33% v/v methyltertbutylether in heptane. The filter cake was dried in a vacuum oven for 12 hours at 45 to 55° C. affording 8.4 kg (72.2%) of the compound of Formula (13) as a solid with an ee of >99.0%.

¹H NMR (400 MHz, CDCl₃); 9.89 (1H, d); 8.56 (1H, d); 7.94 d); 7.22 (1H, d); 5.91 (1H, s,b); 5.58 (1H, s, b); 4.47 (2H, q); 4.43 (3H, s); 3.75 (2H, t); 1.47 (9H, s); 1.19 (9H, s).

### Step 2:

Into a 500 mL three-neck round bottom flask fitted with a mechanical stirrer, an additional funnel and a thermocouple was placed 20 g (39.3 mmol, 1 eq) of compound (13) followed by 60 ml of THF, 20 mL of EtOH, and 100 mL of water. The flask was then charged with 8 mL of 25% sodium hydroxide solution and agitated for about 4 hours while maintaining the the temperature of the reaction mixture at 40° C. Upon judging the reaction complete by HPLC assay, 100 ml of water was added to the reaction mixture and the reaction mixture was heated to 50° C. Once at 50° C., to the reaction mixture was added 30 ml 1N HCl solution over 30 minutes. At the end of the addition period the reaction mixture was stirred for an additional 30 minutes while maintaining the temperature at 50° C., following which another 24 ml 1N HCl solution was added to the reaction mixture over 30 minutes. At the end of the agitation period 60 ml of water was added to the reaction mixture over 30 minutes while continuing to maintain the temperature of the reaction mixture at 50° C. At the end of the addition period the reaction mixture was cooled to room temperature over 1 hour, precipitating a product. The precipitated solids were collected from the reaction mixture by suction filtration and the wet cake collected was washed with 40 ml 1:5 v/v mixed ethanol and water. The solids were dried under vacuum at 60° C. for 12 h affording 16.8 g (90%) of compound (9) as an off white solid.

¹H NMR (400 MHz, _{d6}-DMSO): 9.97 (1H, d), 8.42 (1H, d), 8.20 (1H, d), 7.48 (1H, d), 5.40 (1H, m), 4.07 (3H, s), 1.45 (3H, d), 1.30 (9H, s)

### Step 3:

Part A:

Into a vessel was placed 60 g (184 mmol, 1 eq) (2R, 4S)-4(cyclopropanecarbonyl-amino)-pyrrolidine-1,2-dicarboxylic acid-1-tert-butyl ester 2-ethyl ester (BP) dissolved in 1.2 L EtOAc. This solution was sampled for use as an HPLC 100% standard. The solution was cooled to a temperature of from 20° C. to 35° C. and 36 g (980 mM, 5.3 eq) of HCl(g) was added while maintaining the reaction mixture temperature at a temperature of from 20° C. to 35° C., forming an HCl salt precipitate. When the entire amount of HCl was charged, the reaction mixture was heated to a temperature of from 20° C. to 30° C. and agitated for 1 h. After 1 h, the progress of the reaction was checked for completion by HPLC response of the reaction mixture in comparison with the standard initially sampled. The reaction was continued and sampling repeated until the amount of (BP) relative to standard was ≦0.5% area. The reaction mixture was concentrated under vacuum by distillation with the reaction mixture maintained at a temperature of from 35° C. to 45° C. to a volume of 600 mL, forming a thick slurry. NMP (280 mL) was then added to the reaction mixture and it was further concentrated under the batch under vacuum by distillation with the reaction mixture maintained at a temperature of from 35° C. to 45° C. to a volume of about 560 mL forming a clear solution. This solution was used directly in the Part B coupling step.

### Part B:

Into a 1 L 3-neck round-bottom flask was placed 320 mL of EtOAc, and dissolved therein 80 g of compound (9) (166 mmol, 1 eq), 28 g HOBT.H₂O(182 mmol, 1.1 eq) and 48 g EDCl.HCl (250 mmol, 1.4 eq) in NMP (320 mL). The reaction mixture was stirred for 40 minutes while maintaining the reaction mixture at a temperature of 25° C. The entire amount of the solution of BP prepared in part A (above) was added to the reaction mixture with stirring. The reaction mixture was stirred for 10 min and 80 mL of N-methyl morpholine (724 mmol, 4.4 eq) was added to reaction at a rate which maintained the reaction mixture at a temperature below 35° C. The reaction was monitored by HPLC until a complete reaction was indicated, and 320 mL of EtOAc and 800 mL of water was added to the reaction mixture. The resultant mixture was stirred for 15 min. additional and the layers were separated. The organic layer was washed with 1M HCl (400 mL), followed by 10% K₂CO₃ (400 mL) and then water (400 mL). The organics were concentrated to a volume of 160 mL and 800 mL of acetone was added. The mixture was concentrated to ˜240 mL by distillation under reduced pressure while maintaining the reaction mixture at a temperature of from 40° C. to 50° C. The mixture was diluted with another 800 mL of acetone and again concentrated to a volume of 240 mL by distillation under the same conditions. Following concentration, 800 mL, of heptanes was added to the concentrate while maintaining its temperature at 40° C., precipitating a product. The product solids were collected by filtration and dried under vacuum at 50° C. for 12 h to afford (103 g, 90%) of (10) as an off white solid.

NMR (400 MHz, _{d6}-DMSO): 9.55, 9.03, 8.18, 7.90, 7.77, 7.66, 7.10, 7.04, 6,70, 6.66, 6.10, 5.76, 5.36, 4.91, 4.80, 4.4-3.5, 2.58, 2.30, 1.82, 1.56, 1.47, 1.31, 1.07, 1.001.84, 0.74. Note: due to the presence of rotomers, the observed peaks are listed as observed only.

### Step 4:

Compound (10) (20 g, 29 mmol, 1 eq) was charged to a flask then dissolved in 60 mL of THF, and the solution was cooled to 0-10° C. Concentrated HCl (20 ml) was added slowly to maintain the temperature at 0-20° C. At the end of the charge, the solution was warmed and maintained at a temperature of from 20° C. to 30° C. The reaction mixture was agitated for 4 hours, until the reaction was determined to be complete by HPLC analysis. The reaction mixture was diluted with 2-Me-THF (120 ml) and THF (40 ml) and the reaction was quenched with 20% K₂CO₃ (110 ml) until a pH of 8-8.5 was observed. After adjusting pH, more water (80 ml) was added and the batch was heated to about 30° C. to achieve a clean phase split. The batch was settled for about 15 min, the lower aqueous layer separated, and the organic layer was washed with water (80 ml). The organic phase was diluted with 2-Me-THF (200 ml) and then concentrated under reflux at atmospheric pressure to about 100 ml. The solid product was observed at this volume. The batch was then cooled to 0-10° C. and filtered. The wet cake was washed 2 times with 2-Me-THF (40ml each time). The wet cake was dried for at least 12 h at 60° C. under vacuum affording 13.50 g (79%) of the compound of Formula (Ia) as a white solid.

¹H NMR (spectrum indicates rotomers, only chemical shift is reported, not integration or peak multiplicity; 400 MHz, _{d6}-DMSO) δ 9.82, 9.62, 8.51, 8.38, 8.07, 7.45, 5.46, 4.69, 4.57, 4.33, 4.15, 4.08, 3.99, 3.83, 2.39, 2.26, 2.16, 1.56, 1.44, 1.22, 0.82, 0.69; MSES+ m/z (relative intensity) 590 (M+H).

### Step 5:

Crystalline Form: A solution of The compound of Formula (Ia) (22.0 g, 0.0373 mol) dissolved in hot EtOH (440 ml) was filtered and rinsed with EtOH (44 ml). The solution was heated to reflux and HCl in IPA (5-6N, 10.2 ml) was added. After refluxing for about 30 minutes, the mixture was cooled to about 50° C. and vacuum distilled to about 220 ml. The mixture was then cool to 0° C. over about 50 minutes and agitated for about 30 minutes. The mixture was filtered and washed with EtOH (132 ml). The solid was dried under vacuum at 45° C. to give 20.8 g (89%) of the compound of Formula I as a white solid,

Hydrogen Chloride Salt formation;

A hydrogen chloride salt having a preferred crystalline form can be prepared according to the following method:
a) providing a solution of the compound of Formula Ia in ethanol at reflux;
b) adding HCl in IPA to the solution provided in Step “a”;
c) refluxing the mixture for about 30 minutes;
d) allowing the mixture to cool to about 50° C. and applying vacuum distillation to remove excess ethanol;
e) cooling the mixture to 0° C.; and
f) filtering the mixture and washing and drying to solid to yield the compound of Formula I (a hydrogen chloride salt of the compound of Formula Ia).

### Powder X-Ray Diffraction Sample Preparation

The crystalline form of the compound of Formula I was analyzed as a dry powder for powder x-ray diffraction (“PXRD”) analyses.

### Powder X-Ray Diffraction (PXRD):

X-ray powder diffraction patterns were collected on Rigaku Miniflex Dffractometer with CuK_{α1} source (λ=1.5406 Å) at 30 kV, 15 mA and a solid state detector. A continuous scan was recorded with a step size of 0.02° 2θ and a step time of 2°/min.

Using the method and equipment described above, the compound of Formula I was analyzed by PXRD (FIG. 1) and the characteristic peaks of the powder pattern shown in FIG. 1 are tabulated in Table 1, below. The intensity of the peaks (y-axis is in counts per second) is plotted versus the 2θ angle (x-axis is in degree 2 θ). In Table 2, the four most characteristic peaks from Table I are shown as group 1, the eight most characteristic peaks from Table I are shown as group 2, and the 12 most characteristic peaks from Table I are shown as group 3.

TABLE 1
PXRD Peak Positions for the Compound of Formula I

| Peak Location | Intensity |
|---|---|
| (deg. 2θ) | (Cps) |
| 7.9 | 2073 |
| 8.5 | 848 |
| 9.1 | 151 |
| 10.7 | 587 |
| 13.1 | 263 |
| 13.8 | 1089 |
| 14.2 | 584 |
| 15.6 | 1794 |
| 16.0 | 517 |
| 17.2 | 1189 |
| 18.3 | 1117 |
| 18.5 | 1675 |
| 19.7 | 1068 |
| 20.1 | 307 |
| 20.4 | 222 |
| 20.7 | 203 |
| 21.4 | 512 |
| 21.7 | 629 |
| 22.2 | 182 |
| 22.5 | 172 |
| 23.4 | 3130 |
| 24.4 | 259 |
| 25.4 | 513 |
| 26.3 | 296 |
| 26.7 | 439 |
| 27.2 | 914 |
| 27.6 | 709 |
| 28.4 | 611 |
| 29.3 | 808 |
| 30.5 | 148 |
| 30.9 | 677 |
| 31.3 | 497 |
| 32.0 | 135 |
| 32.5 | 628 |
| 34.2 | 491 |
| 35.3 | 120 |
| 35.9 | 243 |
| 38.4 | 140 |
| 39.0 | 102 |

TABLE 2
Characteristic PXRD Peak Locations for
HCl Salt Form Compound of Formula I

| Peak Location | Peak Location |
|---|---|
| Group Number | (deg. 2θ) |
| 1 | 7.9 |
| | 15.6 |
| | 18.5 |
| | 23.4 |
| 2 | 7.9 |
| | 13.8 |
| | 15.6 |
| | 17.2 |
| | 18.5 |
| | 19.7 |
| | 23.4 |
| | 27.2 |
| 3 | 7.9 |
| | 8.5 |
| | 13.8 |
| | 15.6 |
| | 17.2 |
| | 18.5 |
| | 19.7 |
| | 23.4 |
| | 27.2 |
| | 27.6 |
| | 29.3 |
| | 30.9 |

It will be appreciated exact PXRD peak locations and intensities for a given crystalline form of the same compound will vary within a margin of error depending upon differences in sample preparation, instrumentation, analytical technique, and other factors, however, when these factors are taken into account. PXRD analysis of a compound will yield powder patterns having substantially the same characteristic peaks.

### Differential Scanning Calorimetry (DSC):

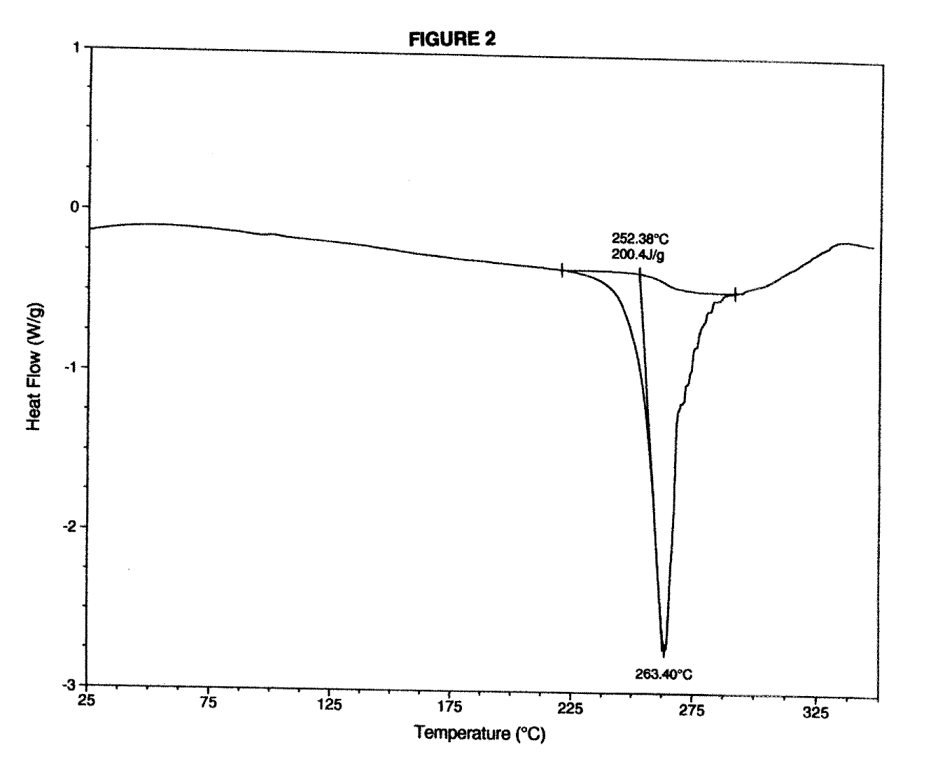

Differential scanning calorimetry data (FIG. 2) was generated with a Q100 Differential Scanning Calorimeter from TA instruments. Samples were sealed in hermetic aluminum pans and two pinholes were punched in the lids of the sample pans. Analysis was conducted under a nitrogen purge with a heating rate of 10° C. per minute.

The heat flow which was normalized by a sample weight was plotted versus the measured sample temperature. The data were reported in units of watts/gram (“W/g”). The plot was made with the endothermic peaks pointing down.

The DSC profile for the crystalline form of the compound of Formula I is shown in FIG. 2. A single melting endotherm was observed with onset temperature of 252° C. and peak temperature of 263° C. The heat of fusion cannot be determined due to the onset temperature of decomposition in TGA data prior to the completion of melting.

The premise for treatment by inhalation is to deliver the drug directly to the site of action (the lungs) with minimal systemic side effects. Therefore, an inhaled compound should exhibit a pharmacokinetic profile with low blood concentration (AUC) due to low oral bioavailability and/or high clearance when given by inhalation or oral dosing routes. It is important that oral AUC be low in order to minimize the effect of any swallowed drug during inhalation.

### Pharmaceutical Compositions

For preparing pharmaceutical compositions from the compound of Formula I, preferably the crystalline hydrogen chloride salt described by this invention, inert, pharmaceutically acceptable carriers can be either solid or liquid. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions for intranasal administration.

Aerosol preparations suitable for inhalation may include solutions and solids in powder form, which may be in combination with a pharmaceutically acceptable carrier, such as an inert compressed gas, e.g. nitrogen.

### Dosages

The quantity of active compound in a unit dose of preparation may be varied or adjusted from about 0.01 μg to about 100 mg, preferably from about 0.01 μg to about 75 mg, more preferably from about 0.01 μg to about 50 mg, and most preferably from about 0.01 μg to about 25 mg, according to the particular application.

The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for inhalation can range from about 0.04 μg/day to about 400 mg/day, in one to four divided doses.

Other than as shown in the operating examples or as otherwise indicated, all numbers used in the specification and claims expressing quantities of ingredients, reaction conditions, and so forth, are understood as being modified in all instances by the term “about.” The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

What is claimed is:

1. The crystalline compound having the structural formula: 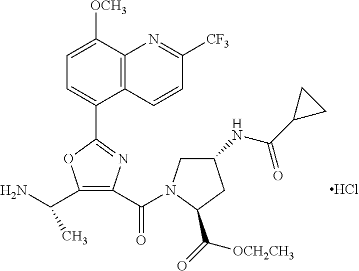
that exhibits a powder x-ray diffraction pattern having characteristic peak locations at 7.9, 15.6, 18.5, and 23.4 degrees 2θ.

2. A method of treating upper or lower obstructive diseases of the airways in a patient in need of such treatment comprising administering to said patient by inhalation an effective amount of the compound of claim 1.

3. The method of claim 2 wherein the disease treated is asthma or chronic obstructive pulmonary disease.

4. A method of treating upper or lower obstructive diseases of the airways in a patient in need of such treatment comprising administering to said patient by inhalation an effective amount of a combination of the compound of claim 1 and at least one additional agent useful for treating upper or lower obstructive diseases of the airway.

5. The method of claim 4 wherein the disease treated is asthma or chronic obstructive pulmonary disease.

6. The method of claim 4 wherein the additional agent is selected from the group consisting of beta-agonists, muscarinic antagonists and corticosteroids.

7. An inhalable pharmaceutical composition comprising an effective amount of the compound of claim 1.

8. An inhalable pharmaceutical composition comprising an effective amount of a combination of the compound of claim 1 and at least one additional agent useful for treating upper or lower obstructive diseases of the airway.

9. A composition of claim 8 wherein the additional agents are selected from the group consisting of beta-agonists, muscarinic antagonists and corticosteroids.

10. A crystalline form of a compound of the formula: 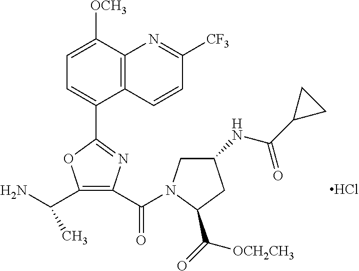
wherein said crystalline form exhibits a powder x-ray diffraction pattern substantially the same as the pattern shown in FIG. 1.

11. The crystalline form of the compound of claim 10 that exhibits a powder x-ray diffraction pattern having characteristic peak locations of 7.9, 15.6, 18.5, and 23.4 degrees 2θ.

12. The crystalline form of claim 11 that exhibits a powder x-ray diffraction pattern having characteristic peak locations of 7.9, 13.8, 15.6, 17.2. 18.5, 19.7, 23.4, and 27.2 degrees 2θ.

13. The crystalline form of claim 11 that exhibits a powder x-ray diffraction pattern having characteristic peak locations of 7.9, 8.5, 13.8, 15.6, 17.2, 18.5, 19.7, 23.4, 27.2, 27.6, 29.3 and 30.9 degrees 2θ.

14. A process for preparing the crystalline form of claim 10 from the compound of Formula Ia 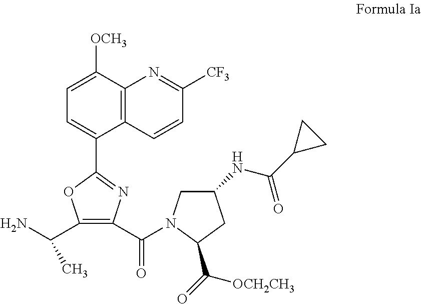
a) combining a mixture of the compound of Formula Ia and hydrochloric acid in a solvent
b) heating the mixture to reflux;
c) adding additional solvent and holding the mixture at reflux for 1 hour allowing the mixture to cool; and
d) filtering the mixture and washing and drying to solid to yield the crystalline form chloride salt of the compound of Formula I.

15. An inhalable pharmaceutical composition comprising the crystalline form of claim 10 and at least one pharmaceutically acceptable excipient or carrier.

16. A purified form of the crystalline form of claim 10.

17. A method of treating upper or lower obstructive diseases of the airways in a patient in need of such treatment comprising administering to said patient by inhalation an effective amount of a crystalline form of claim 10.
